(19) United States
(12) Patent Application Publication
(10) Pub. No.: US 2004/0087997 A1
(43) Pub. Date: May 6, 2004

(54) Medical devices incorporating elastically deformable SIM elements

(75) Inventor: Rodney Brenneman, San Clemente, CA (US)

Correspondence Address: Crockett & Crockett, 24012 Calle De La Plata, Suite 400, Laguna Hills, CA, 92653, US

(73) Assignee: TheraCardia, Inc.

(21) Appl. No.: 10/215,532

(22) Filed: Aug. 9, 2002

(51) Int. Cl. A61M 29/00

(52) U.S. Cl. 606/200

(57) ABSTRACT

Medical devices capable of exhibiting stress-induced martensite, but which do not exhibit stress-induced martensite during deployment or removal. The medical devices are sized and proportioned such that deformation of the device during deployment or removal is elastic, thus avoiding stress-induced martensite during deployment and removal. The use of stress-induced martensite materials decreases the temperature sensitivity of the devices. The SIM materials also allow the medical devices, while in use inside the body, to repeatedly deform and revert to their original shape at constant force without metal fatigue.

### FIELD OF THE INVENTIONS

The inventions described below relate to the field of medical devices. More particularly, the inventions relate to devices comprising materials that may exhibit stress-induced martensite, but are sized and proportioned so that they only elastically deform during deployment or removal.

### BACKGROUND OF THE INVENTIONS

Numerous medical devices incorporating shape memory alloys and pseudoelastic alloys have been proposed, and a few have actually been marketed. Pseudoelastic alloys deform through the formation of stress-induced martensite, if they are deformed beyond their elastic deformation limits, but fully recover their original shape as though the deformation were elastic. Deformation through stress-induced martensite can occur only within a limited temperature range. The alloy must be austenitic to start with, meaning it must be above the martensite range of the alloy, and the temperature must not exceed a certain temperature. The SIM temperature range of the alloy can be adjusted and controlled by alloying and tempering techniques. Medical devices taking advantage of pseudoelastic and SIM behavior are formulated so that they are pseudoelastic at body temperature (that is, they exhibit stress induced martensite behavior in a temperature range which includes human body temperature of 98.6° F. (37° C.)). Pseudoelastic components of medical devices can be deformed substantially beyond their elastic deformation limits through pseudoelastic deformation, without suffering plastic deformation. Plastic deformation is a permanent deformation from which the component cannot recover its original shape. Thus, it is well known that medical devices such as stents, staples, vena cava filters, etc. can be deformed pseudoelastically so that they can be substantially compacted and inserted into a catheter, cannula, or other restraining device, inserted into the body, released from the restraining device, and thereafter fully recover their original shape (subject only to the restraint imposed by body structures at the implantation site). Commercial examples of such devices are the Radius™ coronary stent sold by Boston Scientific and the Vascucoil™ peripheral stent sold by Medtronic. In all pseudoelastic medical devices, nitinol is the preferred SIM alloy because it is readily available, it has proven to be perfectly biocompatible, and is highly resistant to fatigue stress, among other benefits.

Jervis, Medical Devices Incorporating SIM Alloy Elements, U.S. Pat. No. 6,306,141 (Oct. 23, 2001) described several medical devices incorporating SIM alloys. Each of these devices requires formation of stress-induced martensite in the component during insertion of the device into a restraining device prior to insertion into the body. In other words, the component must be pseudoelastically deformed in order to load it into the catheter, cannula, or other restraint. The benefits of doing so are not always necessary, and may induce some unnecessary complexity in loading the component into the insertion device. However, the benefits of pseudoelastic behavior, and other benefits of nitinol, which include very high resistance to fatigue and perfect biocompatibility, can be obtained in an implanted device without the necessity of pseudoelastic deformation during loading into an insertion device. Pseudoelastic components can be deformed elastically, while being stressed with forces insufficient to cause stress induced martensite, if they are formed with an unstressed shaped approximating the implanted shape, and elastically deformed for loading into an insertion device.

### SUMMARY

The devices and methods described below provide for medical devices comprising materials capable of exhibiting SIM, but that are sized and proportioned so that the devices only elastically deform during deployment or removal. In other words, martensite is not formed in the metal due to applied stress either during deployment or during removal. However, the medical devices may exhibit SIM when, after deployment, the devices are in use inside a body. Thus, while in use inside the body the medical devices may be deformed pseudoelastically. The difference between the initial shape, the final shape and the shape required to fit into the insertion device are limited so that deformation for loading may be accomplished merely with elastic deformation of the pseudoelastic component.

### BRIEF DESCRIPTION OF THE DRAWINGS

FIGS. 1 and 2 illustrate the stress-strain behavior of an alloy which exhibits constant stress versus strain behavior due to stress-induced martensite.

FIG. 3 is a side elevation view of a partial section of a catheter which includes a pseudoelastic component in an elastically stressed configuration.

FIG. 4 is a side elevation view of the catheter of FIG. 3 in an unstressed configuration.

FIGS. 5 and 6 show an IUD formed at least partly from a pseudoelastic shape-memory alloy.

FIGS. 7 and 8 shows a pseudoelastic stent suitable for insertion into the body when deformed elastically.

FIGS. 9, 10 and 11 show a pseudoelastic blood clot filter suitable for insertion into the body when deformed elastically.

FIGS. 12 and 13 illustrate occlusive coils suitable for insertion into the body when deformed elastically

FIG. 14 shows an unstressed loop made from a SIM alloy.

FIG. 15 shows the loop of FIG. 14 when elastically stressed.

### DETAILED DESCRIPTION OF THE INVENTIONS

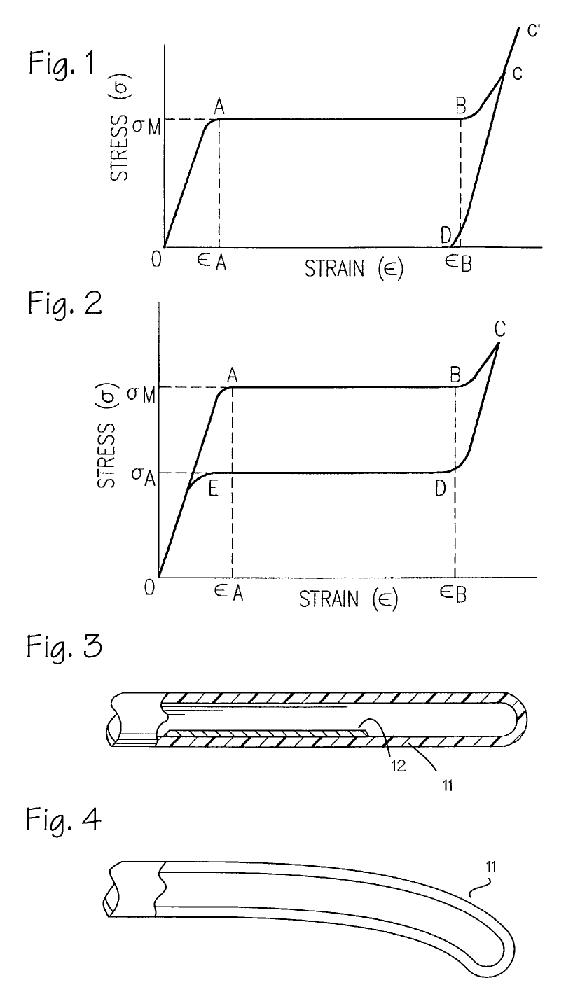

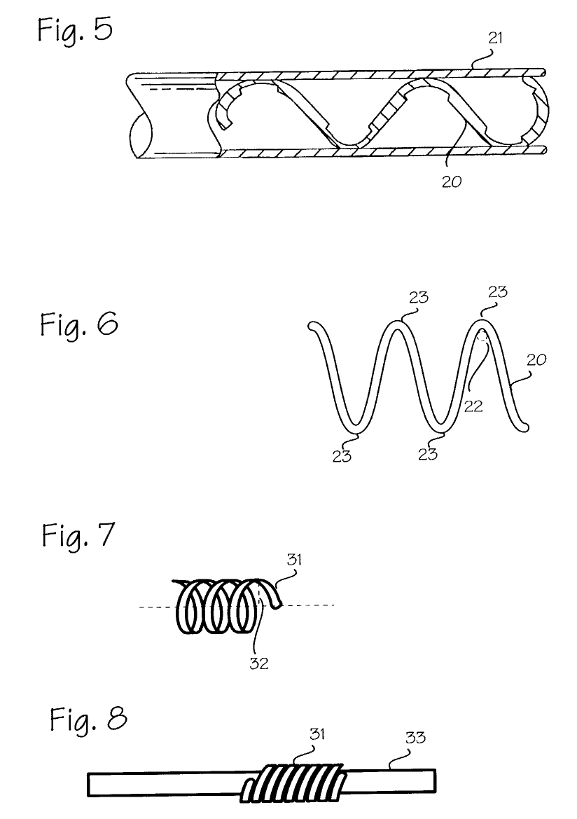

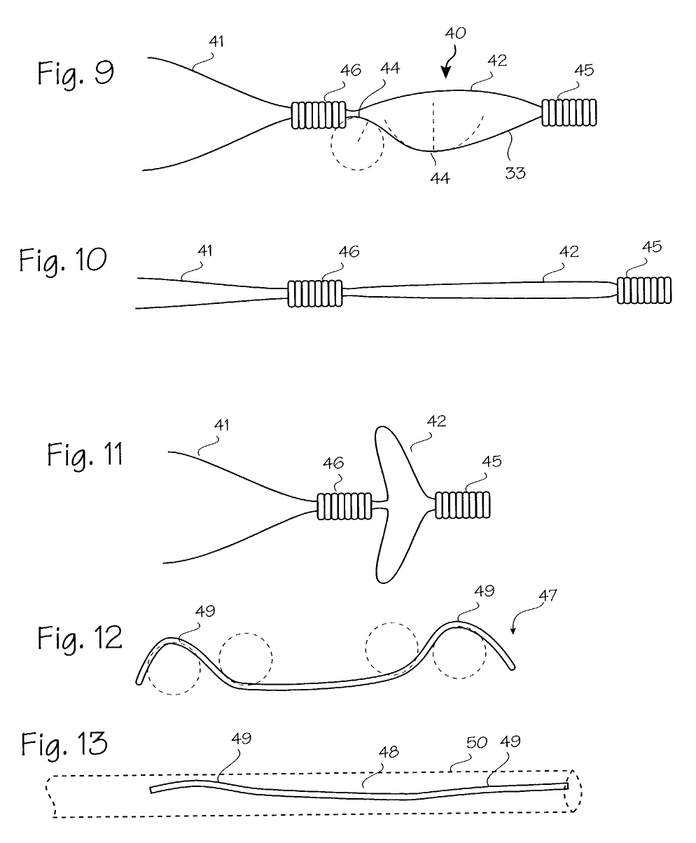

FIGS. 1 and 2 are stress-strain curves illustrating the material behaviors of elastic deformation, pseudoelastic deformation, and plastic deformation in a pseudoelastic alloy such as nitinol. In FIG. 1, the alloy is at a temperature between Mₛ (the temperature at which martensite starts to form during cooling) and M_{d} (the maximum temperature at which martensite may be induced by application of stress), and that it is initially austenitic. FIG. 1 shows the case when the temperature is below Aₛ, (the temperature at which austenite starts to form, upon heating from the martensite state) so that any martensite formed by the applied stress is stable. In FIG. 2, the stress-strain curve relates to an alloy at a temperature above Aₛ, so that austenite is the only stable phase at zero stress, and any martensite formed by application of stress will spontaneously revert to austenite.

In FIG. 1, when a stress is applied to the alloy, it deforms elastically along the line O-A. At a critical applied stress, σₘ, the austenitic alloy begins to transform to (stress-induced) martensite, along line A-B. This transformation takes place at essentially constant stress until the alloy becomes fully martensitic at point B. From that point on, as further stress is applied, the martensite yields first elastically, along curve B-C, and then plastically along curve C-C′. When the stress is released, if released from point C, the martensite recovers elastically to point D, at which there is zero residual stress, but a non-zero residual strain. Because the alloy is below Aₛ, the deformation is not recoverable until heating above Aₛ results in a reversion to austenite. At that point, if the sample is unrestrained, the original shape will be essentially completely recovered through the thermally induced shape memory behavior of the alloy. If the alloy is restrained, it will be recovered to the extent permitted by the restraint. However, if the material is then allowed to re-cool to the original temperature at which it was deformed (or a temperature where SIM behavior of this type is seen), the stress produced in the sample will be constant regardless of the strain, provided that the strain lies within the “plateau” region of the stress-strain curve. That is, for a stress between ε_{B} and ε_{A} the strain will be σₛ. This means that a known, constant force (calculable from σₛ) can be applied over a wide (up to 5% or more for certain Ni/Ti alloys) strain range. Thus, though this resembles the conventional shape memory effect, because the alloy shows SIM and is below Aₛ a constant force can be achieved.

In FIG. 2, which illustrates the stress-strain curve of the alloy when it is at a temperature above Aₛ, when a stress is applied to the alloy, it deforms elastically along line O-A, then by the formation of stress induced martensite (SIM) along line A-B, and by elastic deformation of the martensite to point C, just as in FIG. 1. However, the stress-strain behavior on unloading is significantly different, since the alloy is above Aₛ and the stable phase is therefore austenite. As the stress is removed, the alloy recovers elastically from C to D. Then, at a critical stress, σ_{A}, the alloy reverts to austenite without requiring a change in temperature. Thus, reversion occurs at essentially constant stress along line D-E (meaning that the alloy will exert a constant force on any restraint). Finally if the stress is removed from the reverted austenite, it recovers elastically along line E-O. The formation of stress-induced martensite and the associated reversion of stress-induced martensite to austenite and the austenitic shape is referred to as pseudoelastic or superelastic behavior.

This material characteristics of pseudoelasticity can be very valuable in implanted medical devices. A large degree of deformation (strain) can be imparted, either intentionally or accidentally, and a medical implant or component will revert back to its memorized shape, or exert a constant force against the body in its attempt to revert. Thus for example, a stent which is implanted in a superficial artery or vein such as the carotid artery or the saphenous vein, can be substantially crushed upon impact, but will revert quickly to its tubular shape. In devices intended to exert spring force on body parts, such as staples, venous valve supports, or orthodontic wires, the uniform force exerted over a large range of recovery is quite valuable, since displacements will not increase or decrease the force applied to the body. However, though pseudoelasticity is valuable in a medical device, it is not necessary, and it may be a hindrance if it is used to load the devices in medical devices for insertion into the body. As indicated by the stress-strain curves, the amount of force exerted when a SIM component seeks to recover its original shape (line D-E in FIG. 2) can exceed the force exerted in attempts to recover elastically along the lower portion of line O-A. Thus, for many medical devices, it would be beneficial to provide a pseudoelastic (pseudoelastic at body temperature) component, but load the component into a restraining means through purely elastic deformation, avoiding formation of stress induced martensite.

Catheters and cannulas may be formed of SIM alloys while limiting deformation upon insertion into the body. FIGS. 3 and 4 show a catheter which includes a SIM component configured such that deformation necessary for insertion is limited to elastic deformation. FIG. 3 shows a cross section of the catheter 11 in an elastically stressed configuration. The catheter body is comprised of a pseudoelastic alloy (specifically, the alloy is pseudoelastic at body temperature). A stylet 12 (or a retaining pin, screw, control rod, or other means for retaining the catheter in the straight configuration) is disposed within the catheter to maintain the catheter in the elastically stressed straight configuration. When the stylet is removed, then the catheter will elastically revert to its unstressed, slightly bent configuration. The catheter takes on a slight bend upon removal of the restraining pin, as shown in FIG. 4. Thus, the catheter may be inserted in a straight configuration, but may be easily converted to a slightly bent configuration when desired. If left in place in the body, the catheter body may exhibit pseudoelasticity if it is deformed, whether by movement of the patient, manipulation by the surgeon placing the device, or otherwise. Thus, if the catheter is deformed by stresses inadvertently applied during movement or manipulation of the patient's body, the catheter will revert to its slightly bent shape. The catheter may be removed by inserting the stylet into the catheter as the catheter is removed from the body. As the catheter is removed from the body the catheter elastically bends back to the stressed configuration shown in FIG. 3.

The catheters may be modified for many uses, such as a tracheal puncture catheter. The tracheal puncture catheter should be straight or slightly bent for easy insertion into the trachea through a puncture into the front of the neck, but should curve after insertion so that the flow of air or oxygen through the catheter passes axially down the trachea rather than impinging on the surface of the trachea and damaging it. Thus, a tracheal puncture catheter can be formed in a first, unstressed configuration, in which it is bent a few inches proximal to the distal tip. It may be maintained in a slightly straighter configuration, in which the bend is of lesser angle, with a stylet. Altering the angle of the bend a few degrees, if accomplished over a large radius of curvature, eliminates the possibility of forming stress induced martensite. After the tracheal puncture catheter is inserted into the trachea of the patient and the stylet is remove, it elastically reverts to the unstressed, bent condition. Thereafter, the tracheal puncture catheter may deform pseudoelastically if impacted or inadvertently stressed (as may well happen during patient transport), but will quickly revert to its bent, open tubular configuration when unstressed.

IUD's may be comprised of pseudoelastic alloys, and configured such that the deformation necessary to implant them may be limited to elastic deformation. FIG. 5 shows an IUD 20 disposed within a catheter 21. The IUD comprises an alloy that is pseudoelastic, i.e. that it has the stress-strain curve of FIG. 2 at body temperature, and has an Aₛ that is below body temperature. The IUD may be formed into the desired shape in the austenitic state, being sized and proportioned so that the IUD need only be elastically deformed during deployment or removal. This is accomplished by limiting the curvature and mass of the bend points in the stressed and unstressed configurations. When the placement device is inserted into the uterus, the IUD may be elastically deployed from the placement device, and take on the configuration shown in FIG. 6, in which the bend points revert to the smaller radius bends necessary form the IUD as required for retention. The formation of SIM is avoided during placement. Removal is the reversal of placement: the placement device is inserted into the uterus, the IUD is elastically deformed during withdrawal by the placement device, and the placement device is thereafter withdrawn. The formation of SIM is avoided during placement and removal because the change of radius of curvature 22 of arcs 23 between the stressed state and the unstressed state is limited. However, as the IUD experiences strain within the SIM range, the IUD will tend to revert back to its unstressed form at a constant force (and thus remain in place without damaging the patient).

Stents may be formed of pseudoelastic alloys, and deformed elastically for insertion. If a SIM pseudoelastic wire is used to form the coil, which is then isothermally and elastically deformed by loading into a catheter, then the need for pseudoelastic deformation and/or temperature control is avoided. The wire remains in an elastically stressed state when in the catheter, but elastically re-forms the coil spontaneously when it is extruded from the catheter. The formation of SIM is avoided during placement, though the coil may exhibit SIM while subject to strain inside the body after deployment. FIGS. 7 and 8 illustrate a stent and insertion catheter designed to permit insertion under elastic deformation. The stent 31 is a coiled stent, which in its unstressed state has a predetermined radius 32. The stent may be elastically deformed and restrained on the insertion catheter 33 so long as deformation is uniform along the length of the coil, and the compression is limited to a predetermined compressed radius, which is established by the diameter of the insertion catheter. Compression and deformation limited to the elastic range of the alloy may be achieved for several stent designs, including the illustrated coil, braided stents, and others. Upon release into the body, whether in the vasculature, esophagus, urethra, or elsewhere, will result in elastic expansion of the stent. Thereafter, the stent may be deformed elastically or pseudoelastically, and the patient will obtain the benefit of pseudoelasticity.

Blood clot filters may also be made of pseudoelastic alloys, elastically deformed for insertion, and thereafter deformed within the body as necessary. The filter is formed in the austenitic state, with an Aₛ below body temperature, and sized and proportioned so that the filter may be inserted into the intended blood lumen. The wire is elastically deformed and disposed inside a catheter. The wire is then elastically straightened and inserted into the blood lumen. The filter then elastically re-forms into the filter within the lumen. The formation of SIM is avoided during placement. However, the filter may exhibit SIM while under strain caused by the beating heart.

FIGS. 9, 10 and 11 illustrate a blood clot filter comprising pseudoelastic elements. FIG. 9 shows the blood clot filter 40 in its unstressed state. The anchor wires 41 and filter loops 42 are formed with slightly bowed configuration, and the maximum bend radius at arcs 43 and 44 is limited, so that the device may be straightened as shown in FIG. 10, straightening the arc without stressing the loop to the extent necessary to form stress induced martensite. After release in a blood vessel of the patient, the filter will resiliently revert to the bowed shape of FIG. 9, and can thereafter be deformed to the configuration shown in FIG. 11 by moving the tip 45 proximally toward the collar 46, until the loops are deformed to the extent that they impinge on the walls of the blood vessel at the implantation site.

Guglielmi detachable coils (commonly refereed to as GDC's) and related occlusion devices may also be formed of pseudoelastic alloys, deformed elastically for implantation, and inserted into the body. FIG. 12 illustrates an occlusive coil 47 in its unstressed shape. The coil may include a middle section 48 which is substantially straight, and anchor sections 49 which are curved so as to exert force against a body lumen and hold the coil in place. The coil may also be arcuate and curved along its entire length, as for GDC's intended for placement in an anuerysmal sac. Because the radius of curvature of arcs 50 is limited, relative to the mass of the coil along the arc, it may be straightened and loaded in an insertion catheter 51, as shown in FIG. 13, without the stress needed to induce martensite in the coil. Upon straightening, the small radius of curvature in the unrestrained configuration is opened to a straight configuration (or a very large radius of curvature). The device can be ejected into a lumen of the body. Thereafter, stresses applied by the body, from movement or shrinkage and transformation of vessel, may be resisted by the coil either elastically or pseudoelastically.

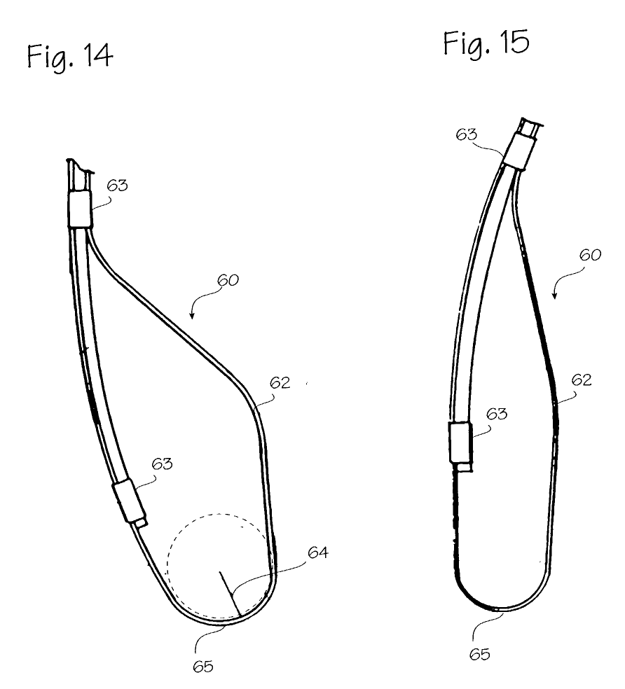

FIGS. 14 and 15 show a strut 60 comprised of a SIM material for use in a cardiac compression device, such as the device described in our co-pending application Ser. No. 10/093,870, filed Mar. 7, 2002 (the entirety of which is incorporated herein by reference). In FIG. 14 the strut is in an unstressed state, but is sized and proportioned so that the loop may be elastically deformed into the state shown in FIG. 15. Thus, the strut may be disposed inside of a cannula and later deployed outside the cannula without inducing SIM in the strut. Later, when already disposed inside the body, the loop may be pseudoelastically deformed during use, exerting a constant level of force against the heart over a wide range of deformation (thus, acting to limit the amount of force applied). To provide for elastic deformation given the structure of the strut, it is comprised of a relatively massive primary member 61 and a slender secondary member 62 which is slidably secured to the primary member with one or more bands 63 which hold the primary member and secondary member together but permit relative sliding along the longitudinal axis of the strut. The secondary member is looped, and the loop has a minimum radius of curvature 64 at the arc 65 so that it may be deformed to the smaller radius of curvature 66 shown in FIG. 15, and inserted into a cannula. Thus, movement into and out of the cannula results in elastic deformation of the secondary member because the deformation of the loop is limited vis-à-vis the unstressed state. Additional bend or arc may be provided in the secondary member to alleviate the total strain at all points along the secondary member, as necessary to permit the total desired deformation to be achieved elastically.

As illustrated in all the embodiments, the medical devices are adapted for insertion into the body and include a component which is pseudoelastic at body temperature. The component is adapted to be constrained to an insertion device (either in or on a catheter or cannula), and has a first configuration when unconstrained and second configuration when constrained to the insertion device. The component has areas of deformation sized and dimensioned such the deformation necessary to change from the first configuration to the second configuration is substantially elastic and devoid of pseudoelastic deformation. The component is deformable by bending an arc on the component, and the bend radius in the first configuration and the bend radius in the second configuration are within a predetermined range which permits deformation from the bend radius in the first configuration to the bend radius in the second configuration through elastic deformation at the bend point. The permissible range of bending, and the change of the bend radius, can be determined empirically, and varies with the cross section of the component, the particular alloy, and the particular transitions temperatures of the alloy. In many of the devices illustrated, strain of up to 1 or 2% (depending on the alloy) can be imposed on the component through purely elastic deformation. If additional compression is required, the component can be formed with additional bend points.

The advantage of limiting deformation of pseudoelastic medical device components to elastic deformation can be employed in a wide variety of additional devices, especially devices such as orthodontic wires, bone plates, bone clamps, tissue staples, stone baskets, TURP tines, etc. for which pseudoelastic components have been proposed. The entirety of all such components can be designed, as illustrated above, to avoid formation of stress induced martensite at all points of deformation, or at select points of deformation (for example, at points where SIM deformation makes the component difficult to handle and load into an insertion device). The design limitations can be applied to nitinol, as well as any other SIM alloy. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. A medical device adapted for insertion into the body, said medical device including a component which is pseudoelastic at body temperature, said component being adapted to be constrained to an insertion device, and having a first configuration when unconstrained and second configuration when constrained to the insertion device, said component having areas of deformation sized and dimensioned such the deformation necessary to change from the first configuration to the second configuration is substantially elastic and devoid of pseudoelastic deformation.

2. The medical device of claim 1 wherein the component is deformable by bending an arc on the component, wherein the bend radius in the first configuration and the bend radius in the second configuration are within a predetermined range which permits deformation from the bend radius in the first configuration to the bend radius in the second configuration through elastic deformation at the bend point.

3. The medical device of claim 1 or 2 wherein the component comprises a stent.

4. The medical device of claim 1 or 2 wherein the component comprises a braided stent.

5. The medical device of claim 1 or 2 wherein the component comprises a stent comprising a helical coil.

6. The medical device of claim 1 or 2 wherein the component comprises a blood clot filter.

7. The medical device of claim 1 or 2 wherein the component comprises an IUD.

8. The medical device of claim 1 or 2 wherein the component comprises a catheter.

9. The medical device of claim 1 or 2 wherein the component comprises a vessel occluding coil.

10. The medical device of claim 1 or 2 wherein the component comprises a vessel occluding coil.

11. A medical device comprising:
an element for use with a target site that is in proximity to a human body such that the element is at human body temperature while in use, the element comprising a shape memory alloy capable of forming stress-induced martensite at human body temperature;
wherein the element is sized and proportioned such that the element is only elastically deformed while being deployed.

12. The medical device of claim 11 wherein the element may form stress-induced martensite when the element is already deployed inside the body.

13. The medical device of claim 11 wherein the element is sized and proportioned such that the element is only elastically deformed while being deployed and while being removed.

14. The medical device of claim 11 wherein the element further comprises an alloy that has an Aₛ temperature below human body temperature.

15. The medical device of claim 12 wherein the element further comprises an alloy that has an Aₛ temperature below human body temperature.

16. The medical device of claim 13 wherein the element further comprises an alloy that has an Aₛ temperature below human body temperature.

17. A medical device comprising:
an element for use with a target site that is in proximity to a human body such that the element is at human body temperature while in use, the element comprising a shape memory alloy capable of forming stress-induced martensite at human body temperature;
wherein the element is sized and proportioned such that the element is only elastically deformed while being deployed;
a restraint holding the element in an elastically deformed configuration, said restraint capable of deploying the element to the target site;
wherein, upon deployment by the restraint, the element elastically reverts towards a preferred shape with respect to the target site.

18. The medical device of claim 17 wherein the element comprises a catheter.

19. The medical device of claim 18 wherein the restraint comprises a pin disposed within the catheter.

20. The medical device of claim 17 wherein the element comprises a filter for a blood vessel.

21. The medical device of claim 17 wherein the element comprises a bone clamp.

22. The medical device of claim 17 wherein the element comprises a wire further sized and proportioned for use as a dental wire.

23. The medical device of claim 17 wherein the element comprises a loop.

24. A medical device comprising:
an element for use within a human body or in such proximity to a human body that the element is at human body temperature while in use, the element comprising a shape memory alloy capable of forming stress-induced martensite at human body temperature;
wherein the element is sized and proportioned such that the element does not form stress-induced martensite while being deployed.

25. The medical device of claim 24 wherein the element may form stress-induced martensite when the element is already deployed inside the body.

26. The medical device of claim 24 wherein the element is sized and proportioned such that the element is capable of being only elastically deformed while being deployed and while being removed.

27. The medical device of claim 24 wherein the element further comprises an alloy that has an Aₛ temperature below human body temperature.

28. The medical device of claim 25 wherein the element further comprises an alloy that has an A_{S} temperature below human body temperature.

29. The medical device of claim 26 wherein the element further comprises an alloy that has an A_{S} temperature below human body temperature.

30. A medical device comprising:
an element for use with a target site that is in proximity to a human body such that the element is at human body temperature while in use, the element comprising a shape memory alloy capable of forming stress-induced martensite at human body temperature;
wherein the element is sized and proportioned such that the element does not form stress-induced martensite while being deployed;
a restraint holding the element in an elastically deformed configuration, said restraint capable of deploying the element to the target site;
wherein, upon deployment by the restraint, the element elastically reverts towards a preferred shape with respect to the target site.

31. The medical device of claim 30 wherein the element comprises a catheter.

32. The medical device of claim 31 wherein the restraint comprises a pin disposed within the catheter.

33. The medical device of claim 30 wherein the element comprises a filter for a blood vessel.

34. The medical device of claim 30 wherein the element comprises a bone clamp.

35. The medical device of claim 30 wherein the element comprises a wire further sized and proportioned for use as a dental wire.

36. The medical device of claim 31 wherein the element comprises a loop.

37. A catheter for use within the human body, said catheter comprising:
a hollow tube, said hollow tube comprising a shape memory alloy capable of forming stress-induced martensite at human body temperature, said hollow tube also being formed into an unstressed configuration; and
a control rod disposed within the hollow tube, said control rod sized and proportioned so that the control rod may force the hollow tube into only an elastically stressed configuration as the control rod is moved along the length of the hollow tube.

38. A catheter for use within the human body, said catheter comprising:
a hollow tube, said hollow tube comprising a shape memory alloy capable of forming stress-induced martensite at human body temperature, and said hollow tube also being formed into an unstressed configuration; and
a control rod disposed within the hollow tube, said control rod sized and proportioned so that the control rod may force the hollow tube into a stressed configuration, without the formation of stress-induced martensite, as the control rod is moved along the length of the hollow tube.

39. A method of deploying a medical device within a human body, said method comprising the steps of:
providing a medical device comprising:
an element for use within a target site that is in proximity to a human body such that the element is at human body temperature while in use, the element comprising a shape memory alloy capable of forming stress-induced martensite at human body temperature;
wherein the element is sized and proportioned such that the element is only elastically deformed while being deployed; and
deploying the medical device to the target site while only elastically deforming the element.

40. The method of claim 39 comprising the further step of removing the medical device from the target site after use, wherein removing the device only elastically deforms the element.

41. A method of deploying a medical device within a human body, said method comprising the steps of:
providing a medical device comprising:
an element for use within a target site that is in proximity to a human body such that the element is at human body temperature while in use, the element comprising a shape memory alloy capable of forming stress-induced martensite at human body temperature;
wherein the element is sized and proportioned such that the element does not form stress-induced martensite while being deployed; and
deploying the medical device without forming stress-induced martensite.

42. The method of claim 41 comprising the further step of removing the medical device from the target site after use, wherein removing the device only elastically deforms the element.

43. A method of using a catheter, said method comprising the steps of:
providing a catheter comprising:
a hollow tube, said hollow tube comprising a shape memory alloy capable of forming stress-induced martensite at human body temperature, and said hollow tube also being formed into an unstressed configuration; and
a control rod disposed within the hollow tube, said control rod sized and proportioned so that the control rod may force the hollow tube into only an elastically stressed configuration as the control rod is moved along the length of the hollow tube; and
using the catheter for a medical procedure within a human body.

44. A method of using a catheter, said method comprising the steps of:
providing a catheter comprising:
a hollow tube, said hollow tube comprising a shape memory alloy capable of forming stress-induced martensite at human body temperature, and said hollow tube also being formed into an unstressed configuration; and
a control rod disposed within the hollow tube, said control rod sized and proportioned so that the control rod may force the hollow tube into a stressed configuration, without the formation of stress-induced martensite, as the control rod is moved along the length of the hollow tube; and
using the catheter for a medical procedure within a human body.
